(19) United States
(12) Patent Application Publication
(10) Pub. No.: US 2014/0127703 A1
(43) Pub. Date: May 8, 2014

(54) Method for Diagnosing Preeclampsia

(71) Applicants: Hans Dieplinger, Innsbruck (AT); Hannes Buchner, Munich (DE); Christian Wadsack, Graz (AT); Ludwig Wildt, Lans (AT); Benjamin Dieplinger, Feldkirchen/Donau (AT)

(72) Inventors: Hans Dieplinger, Innsbruck (AT); Hannes Buchner, Munich (DE); Christian Wadsack, Graz (AT); Ludwig Wildt, Lans (AT); Benjamin Dieplinger, Feldkirchen/Donau (AT)

(73) Assignee: VITATEQ BIOTECHNOLOGY GMBH, Innsbruck (AT)

(21) Appl. No.: 14/127,934

(22) PCT Filed: Jun. 28, 2012
(86) PCT No.: PCT/EP2012/062542

§ 371 Date: Dec. 19, 2013

(30) Foreign Application Priority Data
Jun. 28, 2011 (EP) 11171674.2

(51) Int. Cl. G01N 33/68; C12Q 1/68

(52) U.S. Cl. CPC G01N 33/689; C12Q 1/6883
USPC 435/6.12; 435/7.92

(57) ABSTRACT

Described is a method for in vitro diagnosing whether a pregnant woman has a risk for developing preeclampsia (PE) comprising the steps of determining the afamin content of the pregnant woman in a blood sample or a blood-derived sample, urine, amniotic and cerebrospinal fluid; or determining the content of afamin m-RNA in a liver tissue sample; and comparing the afamin content determined in the sample with a reference value.

The present invention relates to methods for diagnosing preeclampsia.

Physiological pregnancies are generally characterized by increased generation of reactive oxygen species (ROS) due to placental mitochondrial activity and production of superoxide radicals usually accompanied by reduced levels of antioxidants. This condition is called oxidative stress which becomes even more imbalanced in hypertensive pregnancy-associated complications including preeclampsia and HELLP syndrome. Therapeutic applications of antioxidants such as vitamins E and C have therefore been suggested but shown in recent large studies to be essentially ineffective to prevent complications of pregnancy-associated hypertension. Vitamin E is an important lipophilic antioxidant nutrient in the early stages of life, from the time of conception, during pregnancy until the postnatal development of the infant. The mechanisms of its uptake in the placenta and mammary gland seem to depend on lipoprotein receptors as most vitamin E in human plasma is transported via the lipoprotein system.

Preeclampsia (PE) is a multisystem disorder of pregnancy which complicates 3-5% of pregnancies in the western world. It is a major cause of maternal morbidity and mortality worldwide. Major clinical-diagnostic features are hypertension and proteinuria occurring after 20 weeks of gestation in previously normotensive women. The cause of PE remains unknown; the origin of the condition is recognized as lying in the placenta and the only known cure is delivery of the fetus and placenta. A long standing hypothesis has been that PE develops as a consequence of immune maladaption between the mother and the fetus during the first weeks of pregnancy. This process leads to local aberrant feto-maternal immune interaction within the uterine wall and impaired trophoblast invasion of uterine wall and arteries. Subsequently worsened placental perfusion leads then to increased tissue oxidative stress and placental apoptosis and necrosis.

Despite intensive research, risk prediction for PE remains problematic. A marker which identified high-risk women would allow for closer supervision in secondary care. So far, angiogenetic factors such as sFLT-1 and soluble endoglin as well as placental protein 13 (PP-13), pregnancy-associated plasma protein A (PAPP-A), inhibin A and activin A have been reported to predict PE. Various diagnostic marker for PE have been disclosed in WO 2009/097584 A (PE diagnosis by assessing two or more specific markers), WO 2008/046160 A (alpha-1B-glycoprotein), WO 2008/030283 A and US 2006/134654 A (endoglin), WO 2006/034507 A, U.S. Pat. No. 5,108,898 A and U.S. Pat. No. 5,079,171 A (fibronectin), WO 2005/111626 A (ADAM12) and US 2008/233583 A (pappalysin-2). However, most of the currently investigated markers do not appear to have a sufficiently high positive predictive value to be translated into routine clinical practice.

It is therefore an object of the present invention to provide suitable and reliable diagnostic marker for PE or the risk of developing PE, specifically PE markers which discriminate the subjects at risk in the first trimester of pregnancy.

Therefore, the present invention provides a method for in vitro diagnosing whether a pregnant woman has a risk for developing preeclampsia (PE) comprising the steps of

determining the afamin content of the pregnant woman in a blood sample or a blood-derived sample, urine, amniotic and cerebrospinal fluid; or

determining the content of afamin m-RNA in a liver tissue sample; and

comparing the afamin content determined in the sample with a reference value.

In extravascular fluids, such as follicle and cerebrovascular fluid, with limited lipoproteins available, afamin, an alternative carrier protein for vitamin E has been previously described. Afamin is a plasma glycoprotein of the albumin gene family and has been reported to transport vitamin E in vitro and in vivo. It is primarily expressed in liver and secreted into the plasma from where it is transported to the mentioned extra-vascular fluids. While recent work in a cell-culture model of the blood-brain barrier demonstrated afamin-facilitated transport of vitamin E via this barrier, the significance of the vitamin E-binding function of afamin in human fertility remains to be elucidated. Afamin and vitamin E concentrations highly correlate in follicle fluid, but not in plasma. Furthermore, afamin concentrations in follicle fluid also correlate with follicle size and maturity suggesting a general role of afamin in female fertility.

Afamin is a 87 kDa protein belonging to the albumin group and having many things in common, structurally and in terms of biochemistry, with the proteins of this group, such as, e.g., with human serum albumin (HSA), human [alpha]-fetoprotein (AFP) or human vitamin D binding protein. Afamin has already been cloned and sequenced and thus is also available in recombinant form (WO 95/27059 A). Afamin is a glycoprotein primarily of hepatic origin that is secreted into the circulation. It has been shown that afamin occurs abundantly in plasma and other body fluids like follicular fluid, cerebrospinal and seminal fluid. Apart from its sequence homologies to albumin, little is known about the function of afamin. The possibility has been discussed that afamin has sterol binding sites, yet probably does not bind actin. Due to the existing, yet not overwhelming similarity between afamin and albumin, it is doubted that these proteins bind the same ligands (Lichenstein et al., The Journal of Biological Chemistry, 269 (27) (1994), pp. 18149-18154). It has also been shown in vitro and in vivo to possess vitamin E-binding properties (Voegele et al., 2002 Biochemistry 41: 14532-14538).

With the present invention, the role of afamin in physiological human pregnancies was investigated by longitudinal assessments of plasma concentrations of afamin by established ELISA (Voegele et al., 2002) and respective comparisons of afamin plasma values with those of the recognized pregnancy markers hCG+β, hPL and free estriol at different gestational ages. These three markers are synthesized by the human placenta and thus reflect feto-placental growth and development. These results thus served as reference for studies of afamin in pregnancy disorders.

It was shown in the course of the present invention that afamin serum concentrations increased linearly two-fold during the course of healthy pregnancies in two independent Austrian populations. Afamin levels decreased to normal, pre-pregnancy values immediately after delivery. The correlation between afamin concentrations and those of established pregnancy markers such as free estriol, hPL and hCG was negligible to very weak; free estriol and hPL increased and hCG decreased non-linearly, respectively, as described in the prior art. In contrast, to healthy pregnancies, afamin serum concentrations in pregnant patients suffering from PE were significantly elevated already in the first trimester and increased only moderately during the entire time course of pregnancy. In the second, cross-sectional study (see example section of the present specification), PE patients had significantly higher (27%) afamin concentrations compared to controls of the same (first trimester) gestational age (75. 5+10.9 mg/l vs 59.4+13.6 mg/l, p=0.007). Patients with PIH had intermediate afamin levels of 68.7+10.4 mg/l. Expression analysis by RT-PCR and immunohistochemistry revealed no placental afamin expression suggesting exclusive maternal origin of elevated afamin in normal pregnancies.

It could be shown by the study undertaken according to the present invention that afamin is a remarkably predictive marker for PE, especially in the first trimester of pregnancy. Normal plasma values were established in longitudinal assessment pattern and revealed a linear two-fold increase over the pregnancy duration. Two further studies, one longitudinal and one cross-sectional of patients suffering from PE and PIH indicated the suitability of afamin as marker for metabolic disorders unique to the gestational period of life.

Afamin quantification in various body fluids as marker for certain diseases has been disclosed e.g. in WO 2001/001148 A, WO 2002/050549 A, WO 2002/087604 A, WO 2006/079136 A, WO 2009/029971 A and WO 2010/037152 A. Assessment and quantification of afamin in human body fluid and tissue samples is therefore an established tool for certain human medical conditions. These methods are suitable and applicable as well for the purposes of the present invention.

According to the present invention the afamin content of the sample is determined with a suitable afamin determination method and—due to a comparison with an afamin reference—analysed whether the afamin in the sample is increased in comparison with a pregnancy with no risk for PE or not. This can be done e.g. by comparing the afamin content in the sample with an afamin standard, such an afamin reference value from a healthy individual or from an individual not having a risk for developing PE. Alternatively (or in addition), a reference value from a patient with PE or a risk of developing PE is provided. The reference value may be provided e.g. in form of one or more reference samples, reference tables, reference curves or analogous means as well as combinations thereof. In analysing whether the amount in the sample is increased (compared to a healthy status or a “no PE-risk” status), the person skilled in the art has a number of possibilities such as a direct comparison with published reference values of afamin in the body fluid or tissue. In any way, the method according to the present invention does not provide a final medical diagnosis, it provides an afamin value for one sample of unknown PE status or from a person being at risk of or being suspected of having a risk for developing PE compared to an afamin value of a given or virtual sample not having a PE risk. The final medical assessment is then given—independently from the in vitro diagnosing or analytic method according to the present invention—by the individual medically educated person qualified for establishing such diagnosis.

Although all methods for determining afamin are suitable for the present invention, which allow distinguishing between a normal and an increased afamin value (being indicative of a PE risk), the afamin content is preferably determined with anti-afamin antibodies, especially monoclonal antibodies. Such anti-bodies may comprise a detection marker, preferably a chromogenic, fluorogenic or radioactive marker.

The afamin content of a sample is determined according to the present invention to compare this content with an afamin content of a reference value in order to find out whether the afamin content in the sample is increased compared to a “healthy” reference value and therefore could indicate a risk of developing PE or not. The amount detected in the sample is usually expressed relatively to its concentration in blood (e.g. as mg afamin/1 blood) and compared with the afamin amount in blood in women with a “non PE risk pregnancy”. Since afamin content in the blood increases also during healthy pregnancy, it is preferred to use the afamin content of a blood sample of a pregnant woman in the same week of pregnancy who has not developed PE as the reference value. Alternatively, of course also afamin amounts in samples of known and/or confirmed “PE risk” status may be used as a reference sample.

As well, however, tables or figures with reference values may be used as reference values, of course, again with both, “no PE risk” pregnancies and/or “confirmed PE risk” pregnancies.

A preferred embodiment of the present invention therefore relates to a method wherein a risk for developing PE is diagnosed if the afamin content of the sample is increased compared to a reference value of a pregnant woman in the same week of pregnancy who has not developed PE.

Preferably, a risk for developing PE is diagnosed if the afamin content of the sample is increased by 15% or more, preferably by 20% or more, especially by 30% or more, compared to a reference value of a pregnant woman in the same week of pregnancy who has not developed PE.

As an illustrative example according to the present invention, a preferred reference value for not developing PE can be defined as follows:

in weeks 1 to 12 of pregnancy: from 60 to 70 mg afamin/l blood;

in weeks 13 to 16 of pregnancy: from 70 to 77 mg afamin/l blood;

in weeks 17 to 20 of pregnancy: from 77 to 84 mg afamin/l blood;

in weeks 21 to 24 of pregnancy: from 84 to 91 mg afamin/l blood;

in weeks 25 to 28 of pregnancy: from 91 to 98 mg afamin/l blood;

in weeks 29 to 32 of pregnancy: from 98 to 105 mg afamin/l blood;

in weeks 33 to 36 of pregnancy: from 105 to 112 mg afamin/l blood;

in weeks 37 to 40 of pregnancy: from 112 to 119 mg afamin/l blood.

An afamin content which is above such values could indicate a risk for developing PE according to the present invention. Preferably, a risk for developing PE is diagnosed according to the present invention if the afamin content of the sample is increased by 10 mg afamin/1 blood or more, preferably by 15 mg afamin/1 blood or more, especially by 20 mg afamin/1 blood or more, compared to a reference value of a pregnant woman in the same week of pregnancy who has not developed PE.

The present method is specifically suitable for diagnosing PE in the first trimester of pregnancy. Therefore, the blood sample or blood-derived sample is preferably from a pregnant woman in week 1 to 28 of pregnancy, preferably from a pregnant woman in week 1 to 12 of pregnancy.

The method according to the present invention may be combined with any other suitable diagnosing method for PE in order to further assist in verifying and confirming the diagnosis by the medical doctor. The present method may therefore further comprise the determination of additional PE markers in the blood sample or blood-derived sample, preferably the angiogenetic factors soluble fms-like tyrosine kinase-1 (sFltl) and placental growth factor (PGF), as well as placental protein 13 (PP-13), endoglin or combinations thereof.

More specifically, additional PE markers may be determined in combination with the present afamin testing, preferably measurement of blood pressure, determination of protein content in urine, Doppler assessment of uterine artery pulsatility in the first and second trimester, confirmation of smoking, or confirmation of diabetes.

Specifically preferred blood derived samples are those which are typically taken for routine diagnostic purposes, especially a plasma sample, a serum sample or a dried blood spot (Krantz et al., 2011, Prenat. Diagn., DOI 10.1002/pd.2792).

The present method is also specifically suitable to monitor pregnancies. Accordingly, the afamin content can be determined at two or more (three, four, five, six, seven, eight, nine, ten times; e.g. in each month of pregnancy) times and analysed whether the risk for PE is present or not and whether this status is changed (e.g. also under the influence of medicament treatment/prevention of PE in the case of early diagnosed PE risk). The method according to the present invention may therefore repeated at a later stage in pregnancy, preferably for monitoring pregnancy, especially in the first trimester of pregnancy.

According to another aspect, the present invention relates to a kit for performing the method according to the present invention comprising—besides suitable means for determining the amount of afamin in the sample (which are known to a person skilled in the art in principle)—a reference value as defined herein, preferably a reference value of a pregnant woman who has not developed PE or a reference value of a pregnant woman who has developed PE.

The kit according to the present invention may further comprise afamin antibodies, preferably monoclonal afamin antibodies or polyclonal afamin antibodies, secondary labelled antibodies, afamin specific nucleic acids, an afamin-specific enzymatic test, an afamin-specific ELISA, an afamin-specific fluorometric assay or combinations thereof. This embodiment is specifically preferred, if the afamin content is determined by immunological methods, especially if provided in an ELISA format or any other immunosorbent test performed on a solid surface.

According to a further aspect, the present invention relates to the use of a kit for determining the amount of afamin in a sample of a body fluid or in a tissue sample comprising afamin detection means and an afamin reference for diagnosing PE or a risk of developing PE. Kits for determination of afamin are well known in the art (e.g. WO 01/01148 A, WO 95/27059 A or WO 2006/079136 A). Preferably, the use according to the present invention is reduced to practice by applying a method according to the present invention as described above.

Among the usual components of such afamin determination kits, the afamin standard is specifically preferred (e.g. as a standard well in a microtiter ELISA or as standard dot or area on a genechip or protein (antibody) microarray chip.

The invention is further illustrated by the following examples and the drawing figures, yet without to be restricted thereto.

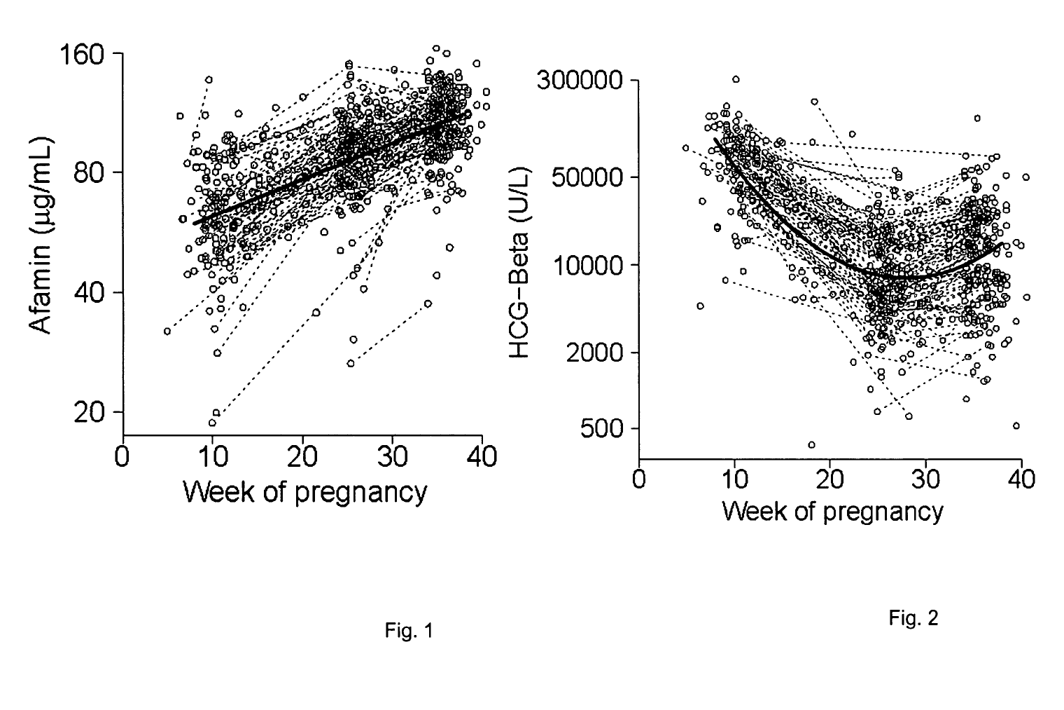

FIG. 1 shows afamin plasma concentrations during healthy pregnancies (Innsbruck subjects). Observed afamin trajectories (grey lines); modelled population mean trajectory (black line);

FIG. 2 shows observed hCG trajectories (grey lines) and modelled population mean trajectory (black line);

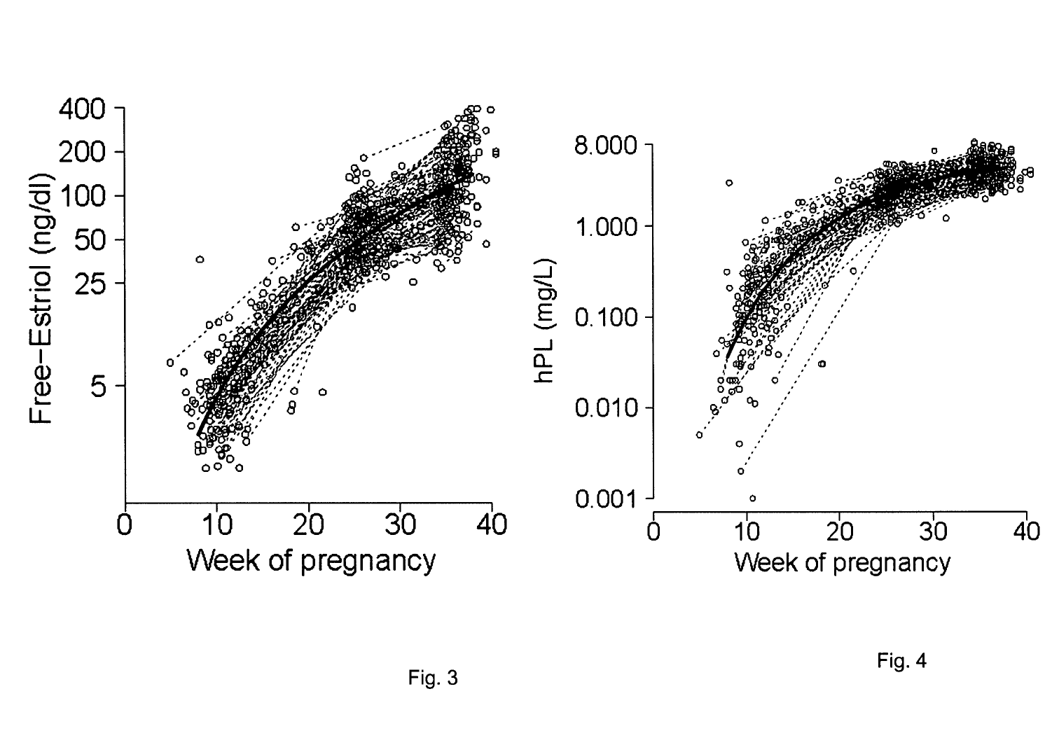

FIG. 3 shows observed free estriol trajectories (grey lines) and modelled population mean trajectory (black line);

FIG. 4 shows observed hPL trajectories (grey lines) and modelled population mean trajectory (black line);

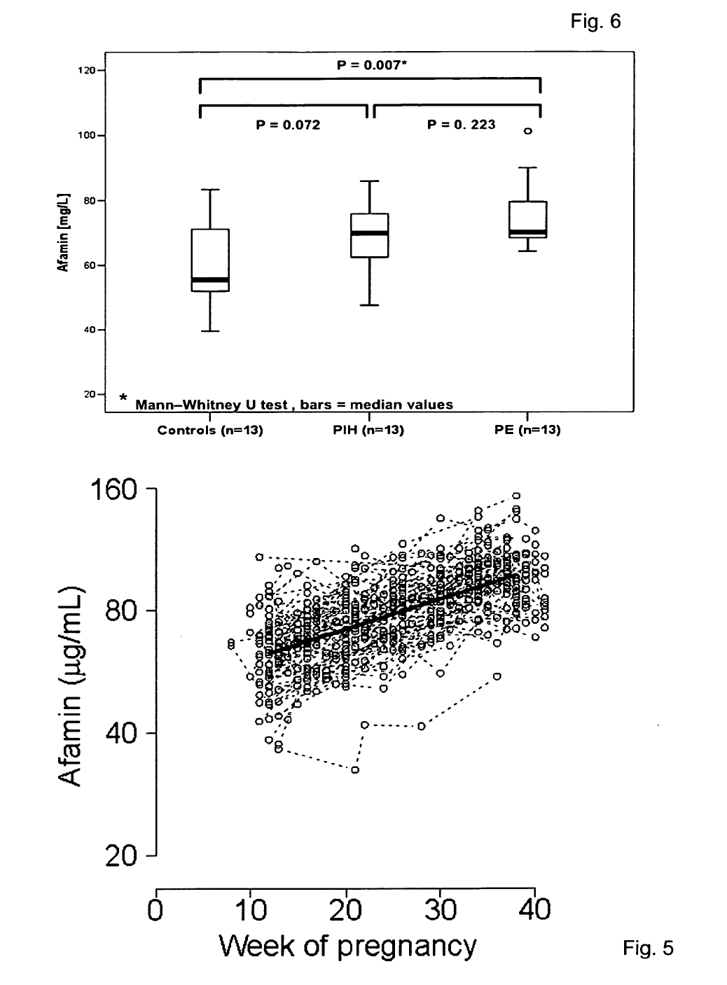

FIG. 5 shows afamin plasma concentrations during healthy pregnancies (Graz subjects). Observed afamin trajectories (grey lines); modelled population mean trajectory (black line;

FIG. 6 shows afamin plasma concentrations in patients with preeclampsia (PE), pregnancy-induced hypertension (PIH) and a healthy control group of pregnant women (n=13 each, samples recruited at comparable gestation week in first trimester);

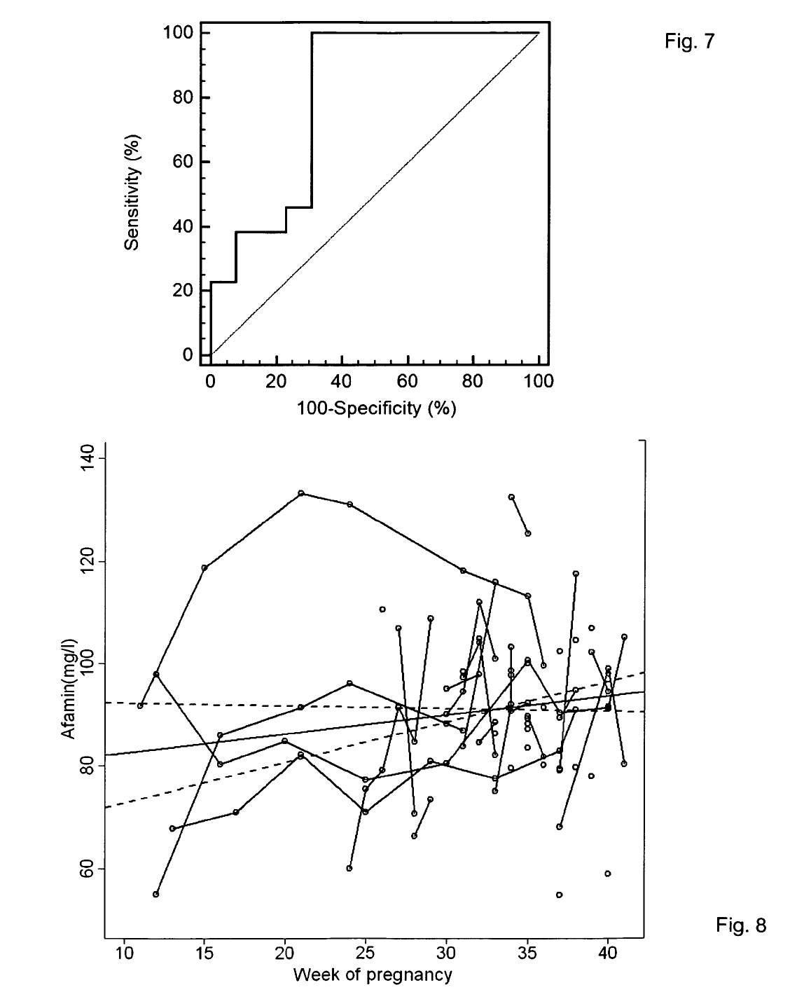

FIG. 7 shows ROC-Plot of Afamin plasma concentrations for the diagnosis of PE (N=26);

FIG. 8 shows afamin plasma concentrations in 48 patients diagnosed with PE (recruited from Gynecol. Department, Graz); The solid red line indicates the average increase of afamin during pregnancy, the dashed red lines show the respective confidence intervals (p=0.119). In 4 patients, afamin could be analysed longitudinally at up to 9 different time points of pregnancy, starting in the first trimester;

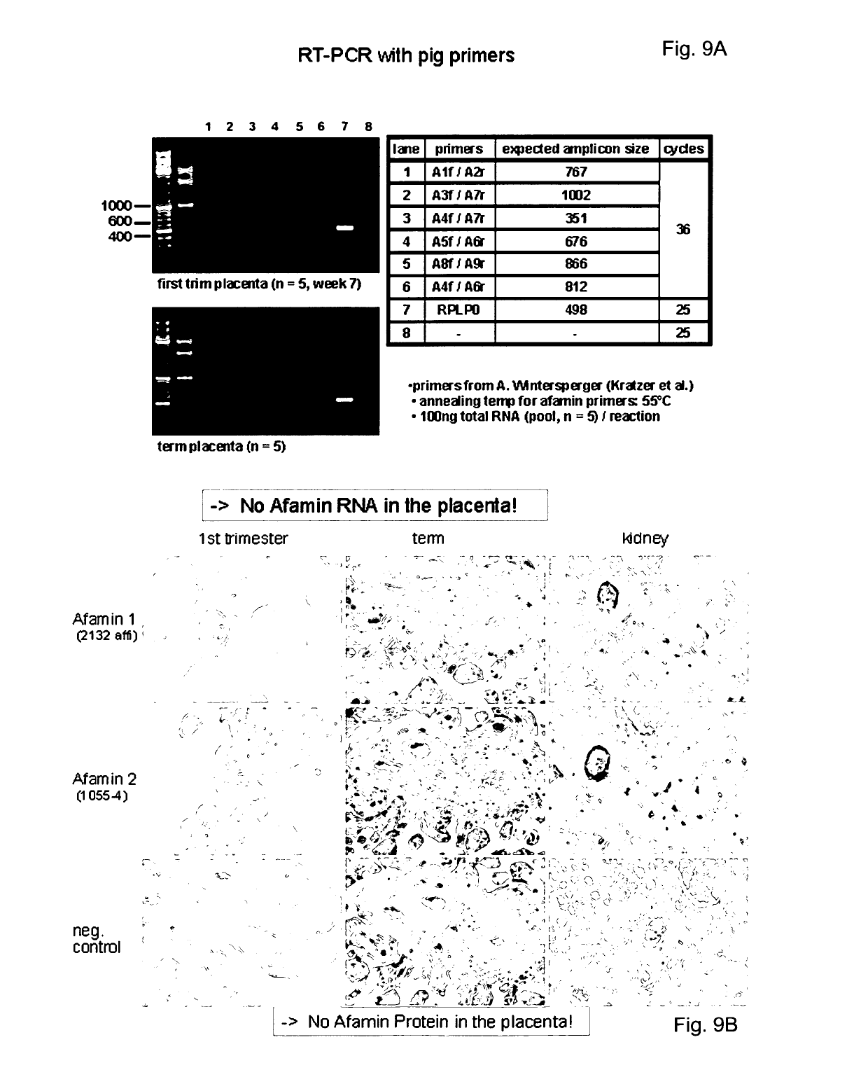

FIG. 9 shows investigation of afamin expression in human placenta on mRNA (A) and protein (B) levels.

### EXAMPLES

### 1.: Study for Determining the Longitudinal Course of Serum Concentrations of Afamin in Pregnancies

### Subjects and Methods

### Subjects

The first study group consisted of a prospective cohort involving a sample size of 467 consecutive pregnant women, aged 14-44 years at delivery, at different gestational ages. Blood was collected of some of those women up to 3 times at different gestational ages but most of them were analysed only once during their pregnancy. All subjects were recruited from the Department of Gynecology and Obstetrics at Innsbruck Medical University, Austria. They were routinely booked at this clinic for the pregnancy.

The second prospective study group of healthy pregnant women was much smaller (n=75) from whom up to 8 blood samples were taken at different gestational age. All these women, aged 19-45 years at delivery, were recruited from the University Clinic of Obstetrics and Gynecology at the Medical University of Graz, Austria. At the time of blood collection all women in both study groups were healthy and had no pregnancy-associated complications.

In the third, cross-sectional study group, serum samples collected from first trimester pregnancies of 3 groups (n=13 each) of women were analysed. 1 group was diagnosed with PE, the other group with PIH and the third group of healthy pregnant women served as controls. 5 patients were diagnosed with PE at <37^{th} week of gestation (weeks 28, 34, 35 and 2 times 36), the remaining 8 patients were diagnosed at >37^{th} week of gestation. Samples and respective clinical data of these patients were collected at the same Clinic in Graz and provided to us by the courtesy of the Institute of Histology at the Medical University of Graz, Austria.

The fourth and last group consisted of 48 pregnant patients diagnosed with preeclampsia recruited at the University Clinic of Obstetrics and Gynecology at the Medical University of Graz, Austria. From 4 patients, up to 9 blood samples, collected longitudinally, spanning almost the entire gestational period, were obtained.

All studies were approved by the local ethics committees and informed consent was obtained from all participants. Blood samples were collected during each visit and serum was prepared from whole blood within 3 hours by low-speed centrifugation. Samples were stored in aliquots of 0,5 ml at −70° C. prior to analysis.

### Biomarker Analysis in Serum Samples

Afamin was determined by previously described double-antibody sandwich ELISA using a biotinylated affinity-purified polyclonal antibody for binding to streptavidin-coated micro-titer plates and the peroxidase-conjugated monoclonal antibody N13 for detection. Both antibodies were raised against afamin purified from human plasma (Vogele et al., 2002).

Free (unconjugated) estriol was measured by competitive enzyme immunoassay using peroxidase-conjugated estriol (which competes with the estriol analyte) and anti-estriol antibody. Human placental lactogen (hPL) was measured by sandwich ELISA using two different monoclonal anti hPL-antibodies. Estriol and hPL assays were purchased from DRG-Instruments (Marburg, Germany) and performed using the liquid handling robotic platform EVO® (Tecan Group Ltd, Mannedorf, Switzerland) and the microplate spectrophotometer Benchmark Plus (Bio-Rad Laboratories, Hercules, Calif., USA).

Human choriongonadotropin (hCG) was measured by sandwich immunoassay on the Modular Analytics Platform E170 (Roche Diagnostics, Mannheim, Germany). This assay quantifies the intact hCG molecule plus the free β-subunit of hCG and is therefore referred to as hCG+β. It uses 2 different monoclonal antibodies against hCG, one of them in biotinylated form to be bound to streptavidin-coated beads, the other one conjugated with ruthenium complex for chemiluminescence detection.

### Afamin Expression Analysis

RT-PCR was performed on mRNA extracted from human first-trimester and term placenta tissue (n=5 each) using 6 different afamin primers according to Kratzer et al. (2009, J. Neurochem. 108: 707-718), with 36 cycles, annealing temperature 55° C. and 100 ng pooled total RNA applied for each reaction. Human RLPO served as endogenous control.

Immunohistochemistry was performed on paraffin-embedded formaldehyde-fixed sections of human placental tissue (first-trimester and term) using 2 different affinity-purified polyclonal anti-afamin antibodies. Sections from human kidney served as positive controls, sections without incubating antibody were negative controls.

### Statistical Analysis

Plasma concentrations of afamin, free estriol, hCG+β and hPL were summarized and compared across trimesters using Analysis of Variance, using logarithm base 2 transformed measures to satisfy the Normality assumptions. Spearman correlations among the markers were computed based on residuals from subtracting a fitted mean (ordinary least squares) from all observations, and then averaging over a moving window with a length of 4 weeks and step width of 0.2 weeks to account for a possibly changing correlation with time. Normal linear mixed models were used to model longitudinal trajectories of individual log base 2 transformed biomarkers over time accounting for within-patient dependencies and potential influence of participant characteristics. The Bayesian Information Criterion (BIC) was used to select the optimal transformations of time for describing the mean trajectory, such as a logarithmic or quadratic transformation of week on pregnancy, which induce a nonlinear trajectory over time, to select which fixed effects influenced the mean trajectory, and to select the number of random effects in the model. All models contained random intercepts to account for within-patient correlation; additional variability of patient trajectories over time, such as random slopes, were tested using likelihood ratio tests. All statistical tests were performed at the two-sided alpha=0.05 level of statistical significance using the R statistical package.

### Results

Table 1 shows the afamin, free estriol, hPL and hCG+β plasma concentrations in 467 females participating in the Innsbruck study accross the three trimesters of pregnancy. Individual patient series and population mean curves over the course of the pregnancy, shown in FIGS. 1-4, depict distinct patterns of individual biomarker concentration changes during the three trimesters of pregnancy (all p-values<0.0001, Table 1). The correlation between all markers was either negligible or very weak: between afamin and free estriol it was −0.04, between afamin and hPL −0.04, between afamin and hCG+β-0.03, between free estriol and hPL 0.26, between free estriol and hCG+β−0.16, and between hPL and hCG+β0.0.

TABLE 1
Plasma concentrations of different pregnancy markers at different gestational ages.

| | Week of pregnancy | | |
|---|---|---|---|
| | First | Second | Third |
| | Trimester | Trimester | Trimester |
| | (1-12) | (13-28) | (≧29) |
| Total N = 665 | N = 119 | N = 283 | N = 263 |
| Afamin (mg/l), Median | 65.07 | 87.83 | 103.70 |
| (25%, 75%-ile) | (52.17, 82.75) | (73.36, 99.03) | (93.00, 118.20) |
| Range | 18.72, 137.30 | 26.40, 150.50 | 37.41, 164.80 |
| free Estriol (ng/dl), Median | 4.42 | 44.30 | 106.10 |
| (25%, 75%-ile) | (2.77, 6.14) | (29.74, 65.58) | (71.88, 156.30) |
| Range | 0.57, 36.39 | 2.08, 179.80 | 2.47, 391.10 |
| hPL (mg/l), Median | 0.127 | 2.151 | 4.054 |
| (25%, 75%-ile) | (0.052, 0.225) | (1.321, 2.838) | (3.189, 4.963) |
| Range | 0.001, 3.004 | 0.020, 4.839 | 0.021, 8.398 |
| hCG + β (U/l), Median | 73640.0 | 9639.0 | 12750.0 |
| (25%, 75%-ile) | (40730.0, 99030.0) | (5309.0, 20900.0) | (5571.0, 22510.0) |
| Range | 4722.0, 304300.0 | 367.8, 201800.0 | 521.4, 147500.0 |

All markers differ between the different trimester (p-value < 0.0001)

Unlike the other established markers of pregnancy, which exhibited a nonlinear course, afamin showed a consistent linear increase during pregnancy (y=0.031x+5.65, average increase of 2.17%, 95% confidence interval (95% CI=2.03% to 2.31%)) per week of pregnancy leading to an approximately doubling of extrapolated average afamin values during the course of pregnancy (FIG. 1). Specifically the linear mixed effects model for the logarithm base 2 transformed afamin course contained an intercept (estimate=5.65, SE=0.05, p-value<0.0001) and slope for the time (estimate=0.031, SE=0.001, p-value<0.0001). There was significant random variation of afamin both at the start of the pregnancy and over the course of time (p-value<0.0001).

In contrast to afamin, hCG+β showed a sharp decline over the first 20 weeks of pregnancies before stabilizing and slightly increasing again (FIG. 2). There was significant patient-to-patient variability in the time course (p-value=0.0003). Of all transformations tested to describe the mean trajectory over weeks of pregnancy, a model containing an intercept term (estimate=20.13, SE=0.25, p-value<0.0001), slope for week (estimate=−0.51, SE=0.02, p-value<0.0001), and a quadratic term for week (estimate=0.009, SE=0.0005, p-value<0.0001) provided the best fit. Free estriol and hPL followed similar trajectories during the course of pregnancy, steeply rising during the first one to two trimesters and only more gradually during the third trimester. There was significant patient-topatient variability in the time course only for hPL (p-value<0.0001) and free estriol (p-value=<0.0001). The mean trajectory for free estriol was described by an intercept (estimate=−6.63, SE=0.25, p-value<0.0001), and slope for the logarithm of time (estimate=3.77, SE=0.08, p-value<0.0001); for hPL the mean trajectory was described by an intercept (estimate=−19.56, SE=0.82, p-value<0.0001), slope for time (estimate=−0.17, SE=0.02, p-value<0.0001), and slope for logarithm of time (estimate=7.80, SE=0.39, p-value<0.0001).

FIG. 5 shows the course of plasma afamin concentrations during healthy pregnancies in the Graz study including 75 females whose blood was investigated at up to 8 different time points of gestational age. The linear increase was very similar (y=0.023x+5.71) compared to the group from Innsbruck and again led to an approximate doubling of afamin levels immediately before delivery compared to basal levels.

In a cross-sectional analysis of data from samples of Graz, serum concentrations of afamin from pregnant women suffering from PE were found to be significantly higher compared to pregnant healthy controls matched for the same gestational age (70, 04 vs 55,39, p=0.007). Eight PE patients delivered their baby at gestational week >37, five patients at week <37. In patients with PIH, a median afamin concentration of 69,75 mg/1 was observed (Table 2, FIG. 6). FIG. 7 shows the ROC plot for afamin for differentiating PE from healthy pregnancies (AUC 0.81 (95% CI, 0.6-0.93)).

TABLE 2
Afamin serum concentrations (mg/l) in preeclampsia (PE),
pregnancy-induced hypertension (PIH) and controls

| | | |
|---|---|---|
| Controls | PIH | PE |

| Nr. | Afamin | Nr. | Afamin | Nr. | Afamin |
|---|---|---|---|---|---|
| 0014 | 62.56 | 0017 | 85.87 | 0013 | 79.46 |
| 0032 | 55.39 | 0031 | 62.34 | 0242 | 66.07 |
| 0111 | 52.23 | 0110 | 69.75 | 0244 | 89.93 |
| 0141 | 39.52 | 0140 | 75.78 | 0342 | 68.45 |
| 0200 | 75.02 | 0158 | 81.30 | 0346 | 74.73 |
| 0243 | 49.85 | 0199 | 60.12 | 0374 | 101.15 |
| 0245 | 52.46 | 0220 | 75.75 | 0381 | 84.68 |
| 0275 | 78.30 | 0228 | 58.10 | 0516 | 68.32 |
| 0343 | 71.07 | 0260 | 68.32 | 0520 | 78.59 |
| 0375 | 51.84 | 0274 | 71.84 | 0541 | 70.04 |
| 0517 | 56.16 | 0282 | 62.86 | 0572 | 64.10 |
| 0573 | 44.08 | 0318 | 73.67 | 0585 | 67.63 |
| 0630 | 83.20 | 0324 | 47.47 | 0631 | 68.80 |
| Median | 55.39 | | 69.75 | | 70.04 |
| 25%-ile | 50.85 | | 61.23 | | 67.98 |
| 75%-ile | 73.05 | | 75.77 | | 82.07 |

Finally, PE patients were also investigated from the study cohort recruited at the Department of Gynecology and Obstetrics in Graz. Altogether, 48 patients were investigated; plasma was collected at up to seven time points: In 4 of the PE patients, the earliest time points of blood collection was within the first trimester of pregnancy (FIG. 8). The solid red line indicates the average increase of afamin during pregnancy (slope=0.369, SE=0.213); the dashed red lines show the respective confidence intervals. Afamin plasma concentrations were, on average, elevated already at the first gestational weeks and, in contrast to the time course in healthy pregnancies, increased only modestly (without reaching statistical significance, p=0.090) during the remaining period of pregnancy.

In order to investigate a possible placental contribution of increased afamin concentrations during pregnancy, afamin expression in human placenta was investigated at the protein level by immunohistochemistry and at the mRMA level by RT-PCR. FIG. 10 clearly demonstrates absence of afamin expression in human placenta.

The present examples demonstrated a linear, approximately two-fold increase of plasma concentrations of the vitamin E-binding protein afamin during the course of normal pregnancies. Thus, plasma afamin levels correlated significantly with gestational age.

The reason for increased circulating afamin concentrations in the maternal blood is completely unclear. Results from our studies of placental tissue expression suggest exclusively maternal origin of afamin, since afamin could not be detected in the placenta by either RT-PCR or immunohistochemistry (FIG. 9). Placental expression and secretion into the maternal circulation has been shown for established pregnancy-related parameters such as estriol, hPL and hCG but also for novel markers such as adrenomedullin. Adrenomedullin is a vasorelaxing peptide; its plasma concentrations increase linearly during pregnancy with gestational age, similar to afamin, but, in contrast to the latter, correlate significantly with placenta-derived hormones such as hPL.

The lacking correlation between afamin plasma concentrations and those of estriol, hPL and hCG is thus in line with the lacking expression of afamin by the human placenta. It is conceivaable that afamin rises during pregnancy due to changing hormonal status and subsequent hormonal regulation of the afamin gene expression in the maternal liver. A comparable mechanism has been reported for hormonal regulation (mostly estrogen-induced) of hepatic synthesis of lipids and lipoproteins leading to physio-logical hyperlipemia during gestation.

An interesting finding of the present study was the linearity of afamin concentrations in correlation with gestational age. This is in considerable contrast to the longitudinal course of hPL, hCG and free estriol which developed in a non-linear mode with increasing gestational age, in accordance with earlier observations.

Most importantly, pregnant women destined to develop PE had significantly higher serum concentrations of afamin in the first trimester compared to gestational-age matched healthy pregnant controls. These increased afamin values did not change significantly until delivery. The reason for these findings is completely unclear but indicates a very suitable marker property for afamin in predicting pregnancy complications such as PE.

### 2.: Comparative Results for Apolipoprotein A-II as a PE Marker Suggested in WO 2009/097584 A

Plasma concentrations of apolipoprotein A-II (mg/dl) were determined in healthy women in each trimester of pregnancy. A comparison was made between healthy pregnant women and patients diagnosed with PE (study cohort from Graz (see above)). Apolipo-protein A-II was measured by immunoturbidimetry using reagents from Greiner Biochemica (Flacht, Germany) and standards from Siemens (Marburg, Germany).

TABLE 3 shows that apolipoprotein A-II is not a suitable PE marker in practice. Apo A-II did neither differ between trimester subgroups nor between respective groups of preeclampsia patients and healthy controls (P=0.731).

TABLE 3

| | Week of pregnancy | | |
|---|---|---|---|
| | First | Second | Third |
| | Trimester | Trimester | Trimester |
| | (1-12) | (13-28) | (≧29) |
| Total N = 524 Healthy | N = 216 | N = 228 | N = 80 |
| ApoA-II (mg/dl), Median | 35 | 36 | 36 |
| (25%, 75%-ile) | (32, 38) | (31, 40) | (30.75, 41) |
| Range | 24, 183 | 16, 50 | 22, 57 |
| Total N = 114 Preeclampsia | N = 11 | N = 26 | N = 77 |
| ApoA-II (mg/dl), Median | 37 | 35 | 35 |
| (25%, 75%-ile) | (30, 40) | (35, 40) | (29, 41) |
| Range | 26, 45 | 23, 52 | 18, 54 |

These data show that apolipoprotein A-II levels in pregnant women do not increase during pregnancy; it is further shown that no difference can be observed between PE patients and healthy controls in all three trimester of pregnancy. The methods and rationale shown in WO 2009/097584 A for PE diagnosis therefore seems to be erroneous as already indicated by earlier results (Rosing et al., 1989, Horm. Metabol. Res. 21: 376-382). It is therefore evident that any results obtained from rough and cursory screening are neither indicative nor relevant for PE diagnosis.

1.-13. (canceled)

14. A method of in vitro detection of a propensity of a pregnant woman to develop preeclampsia (PE) comprising:
determining the afamin content of the pregnant woman in a blood sample or a blood-derived sample, urine, amniotic and cerebrospinal fluid; or
determining the content of afamin m-RNA in a liver tissue sample; and
comparing the afamin content determined in the sample with a reference value.

15. The method of claim 14, wherein the reference value is the afamin content of a blood sample of a pregnant woman in the same week of pregnancy who has not developed PE.

16. The method of claim 14, wherein a risk for developing PE is diagnosed if the afamin content of the sample is increased compared to a reference value of a pregnant woman in the same week of pregnancy who has not developed PE.

17. The method of claim 14, wherein a risk for developing PE is diagnosed if the afamin content of the sample is increased by 15% or more compared to a reference value of a pregnant woman in the same week of pregnancy who has not developed PE.

18. The method of claim 17, wherein a risk for developing PE is diagnosed if the afamin content of the sample is increased by 20% or more compared to a reference value of a pregnant woman in the same week of pregnancy who has not developed PE.

19. The method of claim 18, wherein a risk for developing PE is diagnosed if the afamin content of the sample is increased by 30% or more compared to a reference value of a pregnant woman in the same week of pregnancy who has not developed PE.

20. The method of claim 14, wherein a reference value for not developing PE is:
in weeks 1 to 12 of pregnancy: from 60 to 70 mg afamin/l blood;
in weeks 13 to 16 of pregnancy: from 70 to 77 mg afamin/l blood;
in weeks 17 to 20 of pregnancy: from 77 to 84 mg afamin/l blood;
in weeks 21 to 24 of pregnancy: from 84 to 91 mg afamin/l blood;
in weeks 25 to 28 of pregnancy: from 91 to 98 mg afamin/l blood;
in weeks 29 to 32 of pregnancy: from 98 to 105 mg afamin/l blood;
in weeks 33 to 36 of pregnancy: from 105 to 112 mg afamin/l blood;
in weeks 37 to 40 of pregnancy: from 112 to 119 mg afamin/l blood.

21. The method of claim 14, wherein a risk for developing PE is diagnosed if the afamin content of the sample is increased by 10 mg afamin/1 blood or more compared to a reference value of a pregnant woman in the same week of pregnancy who has not developed PE.

22. The method of claim 21, wherein a risk for developing PE is diagnosed if the afamin content of the sample is increased by 15 mg afamin/1 blood or more compared to a reference value of a pregnant woman in the same week of pregnancy who has not developed PE.

23. The method of claim 22, wherein a risk for developing PE is diagnosed if the afamin content of the sample is increased by 20 mg afamin/1 blood or more compared to a reference value of a pregnant woman in the same week of pregnancy who has not developed PE.

24. The method of claim 14, wherein the blood sample or blood-derived sample is from a pregnant woman in week 1 to 28 of pregnancy.

25. The method of claim 24, wherein the blood sample or blood-derived sample is from a pregnant woman in week 1 to 12 of pregnancy.

26. The method of claim 14, wherein the method further comprises determination of additional PE markers in the blood sample or blood-derived sample.

27. The method of claim 26, wherein the additional PE markers in the blood sample or blood-derived sample are the angiogenetic factors soluble fms-like tyrosine kinase-1 (sFltl) and placental growth factor (PGF), as well as placental protein 13 (PP-13), endoglin, or a combination thereof.

28. The method of claim 14, wherein the method further comprises determination of additional PE markers.

29. The method of claim 28, wherein the additional PE marker is determined by measurement of blood pressure, determination of protein content in urine, Doppler assessment of uterine artery pulsatility in the first and second trimester, confirmation of smoking, and/or confirmation of diabetes.

30. The method of claim 14, wherein the blood-derived sample is a plasma sample, a serum sample or a dried blood spot.

31. The method of claim 14, further comprising repeating the method at a later stage in pregnancy.

32. The method of claim 31, wherein the method is completed at least twice during a first trimester of the pregnancy.

33. A kit for performing the method of claim 14, comprising a reference value.

34. The kit of claim 14, wherein the reference value is a reference value of a pregnant woman who has not developed PE or a reference value of a pregnant woman who has developed PE.

35. The kit of claim 33, further comprising afamin antibodies, secondary labelled antibodies, afamin specific nucleic acids, an afamin-specific enzymatic test, an afamin-specific ELISA, an afamin-specific fluorometric assay or a combination thereof.

36. The kit of claim 35, wherein the afamin antibodies are monoclonal afamin antibodies or polyclonal afamin antibodies.
